(19) United States
(12) Patent Application Publication
(10) Pub. No.: US 2005/0112567 A1
(43) Pub. Date: May 26, 2005

(54) Serological assay for Neospora caninum

(75) Inventors: David A. Brake, East Lyme, CT (US); Dianne M. Ritter, East Greenwich, RI (US)

Correspondence Address: SCULLY SCOTT MURPHY & PRESSER, 400 GARDEN CITY PLAZA, GARDEN CITY, NY, 11530, US

(73) Assignee: Pfizer Inc.

(21) Appl. No.: 10/283,540

(22) Filed: Oct. 30, 2002

Related U.S. Application Data
(60) Provisional application No. 60/334,811, filed on Oct. 31, 2001.

(51) Int. Cl. C12Q 1/68

(52) U.S. Cl. 435/6

(57) ABSTRACT

The present invention relates to the serological detection of Neospora caninum-vaccinated animals by means of reaction of a subject serum with a protein reactive with N. caninum. The protein may be a full-length native or recombinant N. caninum or Toxoplasma gondii bradyzoite fusion protein or a truncated fusion protein or fragment thereof. The protein may be used in any of a number of assays including enzyme linked immunosorbant assays (ELISA), a radioimmunoassay (RIA), a Western Blot and other suitable forms of immunoassay.

### FIELD OF THE INVENTION

The present invention relates to the serological detection of Neospora caninum infection by means of reaction of a subject serum with a protein reactive with N. caninum.

### BACKGROUND OF THE INVENTION

Neospora caninum and Toxoplasma gondii are closely related protozoan parasites responsible for disease in a wide number of animals. N. caninum has been recognized as a cause of abortion, neurologically-associated limb defects and neonatal mortality in cattle and other animals. T. gondii is a common cause of ovine abortion. Effective management of neosporosis and toxoplasmosis requires rapid diagnosis. The development of efficient diagnostic tests for N. caninum is therefore critical.

There are several art-recognized tests available to determine whether or not an animal has been exposed to N.caninum. However, diagnostic tests and methods for distinguishing between N. caninum naturally seropositive animals and N. caninum vaccinated and/or N. caninum seronegative (unvaccinated) animals have heretofore not been developed.

### SUMMARY OF THE INVENTION

The present invention, for the first time, provides a method for distinguishing a N. caninum naturally seropositive animal from an animal vaccinated with a Neospora vaccine. The present invention also provides a method for determining whether or not an animal has been infected with N. caninum using a serological assay.

One aspect of the present invention provides a method for detecting antibodies to Neospora caninum in a serum sample by contacting the serum with a Toxoplasma gondii protein and examining the sera for the presence of bound antibody.

### BRIEF DESCRIPTION OF THE FIGURES

FIG. 1 shows Western blot reactivity against truncated BAG1-GST

Lane 1: rabbit antisera raised against full-length BAG1-GST (positive control)

Lane 2: serum from naive, seronegative cow (negative control)

Lane 3: day 49 pooled antisera from N. caninum killed vaccine immunized cows

Lane 4: day 49 pooled antisera from N. caninum attenuated vaccine immunized cows

Lane 5: pooled antisera from naturally infected seropositive cows

Lane 6: MW markers (kDa)

FIG. 2 shows Western blot reactivity against truncated and full-length BAG1-GST

Lanes 1-4: truncated BAG1-GST antigen

Lane 5: MW markers (kDa)

Lanes 6-9: full-length BAG1-GST antigen

Lanes 1,6: serum from naive, seronegative cow (negative control)

Lanes 2, 7: day 119 pooled antisera from N. caninum killed vaccine immunized cows

Lanes 3, 8: day 119 pooled antisera from N. caninum attenuated vaccine immunized cows

Lanes 4, 9: pooled antisera from naturally infected seropositive cows

Lane 5: MW markers (kDa).

### DETAILED DESCRIPTION OF THE INVENTION

The present invention provides an assay system which is able to distinguish between vaccinated and naturally exposed Neospora-seropositive animals. In accordance with the present invention, proteins from Toxoplasma gondii and N. caninum have been found to be present only in naturally seropositive animals. N. caninum seronegative animals and animals which have been previously vaccinated with Neospora-based vaccines (see, e,g. U.S. Ser. No. 09/260,414 “Attenuated Live Neospora Vaccine”, incorporated herein by reference and U.S. Ser. No. 09/138,985 “Neospora Vaccine”, incorporated herein by reference) can now be reliably identified and sequestered from seropositive (i.e. naturally exposed) animals based on the assay results provided in accordance with the methods of the present invention.

Sera from animals exposed to N.caninum will react with Toxoplasma gondii and N. caninum proteins and fragments thereof. By “react” is meant a detectable antigen-antibody complex will be formed. The formation of an antigen-antibody complex is indicative of an animal naturally infected with N. caninum. Sera from animals previously vaccinated with N. caninum and sera from animals naturally seronegative for N. caninum will not react with Toxoplasma gondii and N. caninum proteins and fragments thereof. Accordingly, the invention provides a method to distinguish between vaccinated (seronegative) and naturally exposed Neospora caninum-seropositive animals

Moreover, in accordance with the present invention, N. caninum seropositive animals can be vaccinated based on the results of the assay of the present invention, if desired. Animals contemplated by the present invention include cattle, calves, bulls, cows and steer. Preferably, the method of the present invention is applied to cows and most preferably to a calf.

In accordance with the present invention, any purified, native or recombinant bradyzoite antigen or fragment thereof from Toxoplasma gondii or Neospora caninum, can be employed as a detection antigen for the serological assay of the invention. By “fragment” is meant a peptidic portion of the full-length T. gondii or N caninum protein which provides an epitope which is recognized by antibodies present in animal sera. Truncated forms of the full-length T. gondii and N caninum proteins are also understood to be fragments, in accordance with the present invention.

In one embodiment, full-length bradyzoite antigen glutathione-S-transfersase (GST) fusion protein (BAG-1) is the detection antigen, (SEQ ID NO. 2). In another embodiment, a truncated BAG-1-GST fusion protein, lacking the N-terminal 28 amino acids of BAG1 is the detection antigen, (SEQ ID NO. 3). BAG-1 is also known in the art as BAG-5. The present invention contemplates that antisera from an animal naturally infected with N. caninum or antisera from an animal vaccinated with N. caninum or uninfected antisera can be employed as the test sample.

The reactivity of a bradyzoite antigen or fragment thereof when immobilized on a solid support provides a method of detecting N. caninum antibodies in accordance with the present invention. Specifically, the methods of the invention can be conducted by contacting a sample of animal serum with the bradyzoite-stage protein or fragment thereof, which can then react to form an antigen-antibody complex, and examining the resulting complex for reaction by a conventional detection and quantitation methods. By “antibody” is meant an immunoglobulin molecule able to bind to a specific epitope on an antigen. Antibodies can be a polyclonal mixture or a monoclonal. Antibodies can be intact immunoglobulins derived from natural sources or from recombinant sources and can be immunoreactive portions of intact immunoglobulins.

Numerous methods of examining the quantity and/or the presence or absence of the antigen-antibody complex are known and all are contemplated by the present invention. For example, a Western blot assay may be performed by attaching the antigen to a solid support, such as nitrocellulose paper, incubating the paper with a serum test sample, and examining the nitrocellulose paper for the presence or absence of a specific band at the expected molecular weight for the full length (about 55 kDa) or truncated (about 51 kDa) T. gondii BAG1-GST protein. The presence of a band at either of the expected molecular weights indicates that the animal is seropositive for N. caninum. Conversely, the absence of a band at either of the expected molecular weights indicates that the animal is vaccinated.

The present invention also contemplates a Western blot assay wherein the antigen is attached to a nitrocellulose paper and the antigen is stained with an antibody which has a dye attached. An enzyme-linked immunosorbant assay (ELISA) can also be employed where the antigen or antibody is labeled with an enzyme. The present invention also contemplates a radioimmunoassay wherein the antigen or antibody is labeled with a radioactive element.

The present invention contemplates a method of examining animal serum for the presence/quantity or absence of N. caninum antibodies. Thus, a diagnostic assay kit containing the assay and controls for positive and/or negative reactions, with other necessary ingredients can be assembled in accordance with the teachings of the present invention.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLE1

Antisera from N. caninum seronegative, N. caninum Vaccinated Animals

Any serum sample from a seronegative or PCR negative animal that subsequently receives a N.caninum tachyzoite based modified-live, killed or subunit-based vaccine can be used. In this example, four-month old calves were determined to be seronegative by a diagnostic ELISA test conducted by a qualified veterinary diagnostic lab (Washington State Diagnostic Lab, Pullman, Wash.). Calves were vaccinated on day 0 and day 21 with either a tachyzoite-based i) modified-live, attenuated Neospora vaccine (U.S. patent application Ser. 09/260,414 “Attenuated Live Neospora Vaccine”, incorporated herein by reference) or ii) inactivated, whole cell homogenate Neospora vaccine (U.S. patent application Ser. No. 09/138,985 “Neospora Vaccine”, incorporated herein by reference). Both vaccines are based on tachyzoite antigens as the protective component of the vaccine. The N. caninum modified-live, attenuated vaccine contained 5×10⁷ Ncts-8 tachyzoites per dose and was administered subcutaneously to 3 seronegative animals. The N. caninum inactivated homogenate vaccine contained 100 ug total tachyzoite protein/dose, formulated in a saponin-based adjuvant (750 ug Quil A/150 ug cholesterol) and was administered subcutaneously to 3 seronegative animals. On days 49 and 119 post-vaccination, serum from the three calves in each vaccine group were collected, pooled and frozen in aliquots at −20° C. until testing in the serological assay (see Example 4).

### EXAMPLE 2

Antisera from N. caninum Naturally Infected, Seropositive Animals

Any serum sample from a naturally infected, seropositive or PCR positive animal can be used. In this example, the serological status of individual animals from a commercial cattle herd was determined using a diagnostic ELISA test conducted by a qualified veterinary diagnostic lab (Washington State Diagnostic Lab, Pullman, Wash.). Twenty-one animals reported as seropositive by this test were identified. A serum sample from each seropositive animal was collected, samples pooled, and frozen in aliquots at −20° C. until testing in the serological assay (example 4).

### EXAMPLE 3

Bradyzoite Antigen Used in Neospora Serological Test

Any purified, native or recombinant Neospora caninum or Toxoplasma gondii bradyzoite protein can be used (T. gondii bradyzoite protein BAG1; Genebank Accession No: U23944). In this example, two different recombinant produced forms of the T. gondii bradyzoite antigen, BAG1 (also known in the art as BAG5) were used. The first form was a full-length BAG1-glutathione-S-transferase (GST) fusion protein expressed and purified from E. coli using affinity chromatograph (S. F. Parmley, et. al. 1995. Mol. Biochem. Parasit. 73: 253-257, incorporated herein by reference). The second form was a truncated BAG1-GST fusion protein, lacking the first 28 amino acids, expressed and purified from E. coli using affinity chromatography (Parmley et al., 1995). Purified, recombinant full-length BAG1-GST and truncated BAG1-GST proteins were stored frozen at −20° C. until testing in the serological assay (example 4).

### EXAMPLE 4

Serological Test

Any antibody-based detection test (ELISA, competitive ELISA, RIA, Western blot, etc.,) known in the art can be used. In this example, a Western blot assay was employed. A total of 5 mg T. gondii full-length BAG1-GST or truncated BAG1-GST recombinant protein was thawed, mixed with 5X denaturing sample loading buffer (Pierce, Ill.) and loaded onto a 1 mm×2 well, precast 4-20% Tris-glycine continuous gradient gel (Novex, San Diego, Calif.). The gel was run according to manufacturer's specifications at 125V for 1.5 hrs. The gel was blotted onto a 20 uM nitrocellulose sheet (Bio-Read, Hercules, Calif.) at 25V for 1 hr. The sheet was dried and cut into equal length/width strips. The strips were incubated overnight at room temperature in a blocking solution (5% skim milk in phosphate-buffered saline (PBS)), rinsed in PBS and then incubated with a serum test sample (from example 1 or 2 above).

Aliquots of serum test samples from example 1 and 2 were thawed at room temperature and a freshly prepared 1:200 dilution in PBS was made. Serum from a naive, seronegative cow was used a negative control (1:200 dilution).Rabbit antisera against full-length BAG1-GST was used as a positive control (received as gift from M. McAllister; M. McAllister, et. al., 1996. J. Parasitol. 82(2): 354-355.) and was diluted 1:12,500 in PBS prior to use.

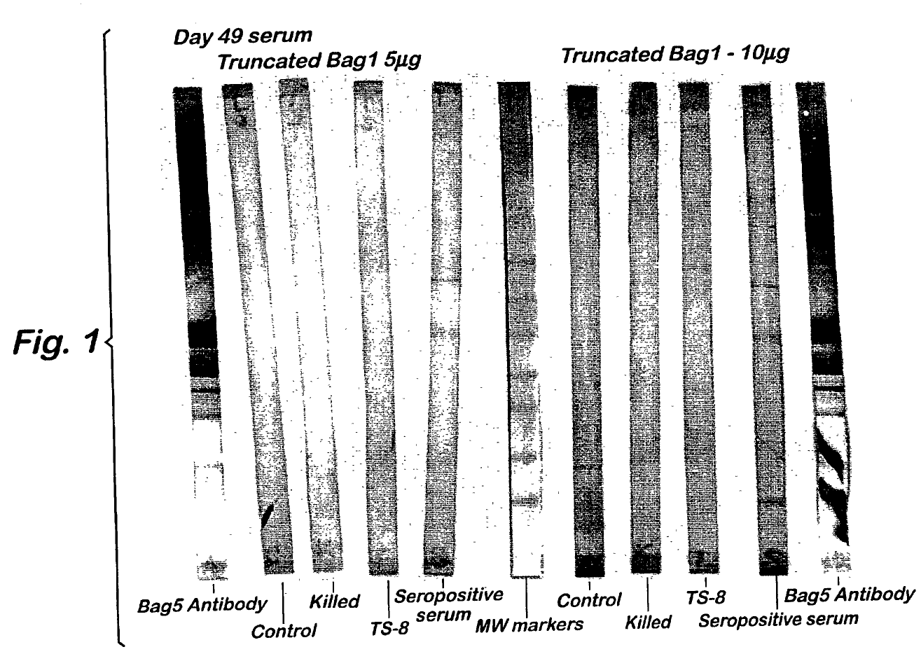

An individual test strip (prepared in example 3) was added to each diluted serum sample and incubated for 1 hr. at room temperature. The strips were briefly rinsed two times in PBS containing 0.05% Tween 20 (PBST) and added to a solution containing a 1:400 dilution (in PBS) of affinity purified phosphatase labeled goat anti-bovine IgG (KPL, Gaithersburg, Md.). Following a 1-hr incubation at room temperature, the strips were briefly rinsed two times in PBST and then incubated in a phosphatase substrate, BCIP/NBT (KPL, Gaithersburg, Md.). After the appropriate development of color (approximately 10 min) the enzymatic reaction was terminated by briefly placing the strips in distilled water. The strips were then air-dried. Results are shown in FIG. 1.

### EXAMPLE 5

Interpretation of Serological Test

A positive serological test is indicated by the presence of a specific reaction between the serum test sample and the N. caninum or T. gondii bradyzoite antigen. In this example, the strips used in Example 4 were examined for a specific reaction by determining the presence or absence or a specific band at the expected molecular weight for the full-length (approximately 55 kDa) or truncated (51 kDa) T. gondii BAG1-GST protein.

As shown in FIG. 1, lane 5, an approximately 52-55 kDa band was present in the strip incubated with the pooled sera from N. caninum naturally, infected cattle, indicating a specific reaction between the N. caninum naturally, infected seropositive test sample and the truncated BAG1-GST bradyzoite antigen. In contrast, in lanes 3 and 4 of FIG. 1, no reactivity against the 55kDa BAG 1-GST protein was detected, indicating the lack of a specific reaction using either of the N. caninum vaccinated test samples.

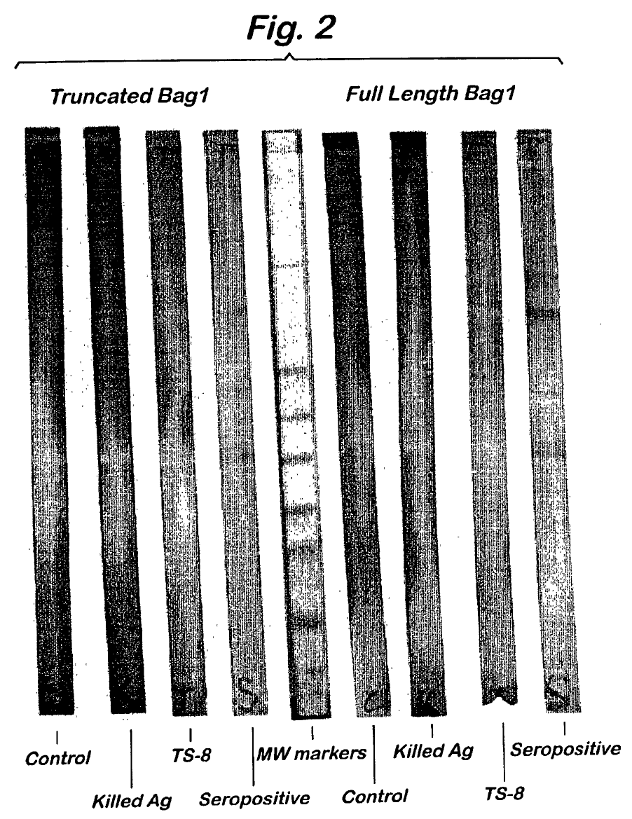

As shown in FIG. 2, lanes 4 and 9, an approximately 52-55 kDa band was present in the strips incubated with the pooled sera from N. caninum naturally, infected cattle, indicating a specific reaction between the N. caninum naturally, infected seropositive test sample and the truncated (lane 4) or full-length (lane 9) BAG1-GST bradyzoite antigen. In contrast, in lanes 2 and 7 of FIG. 2, no reactivity against either form of the BAG1-GST protein was detected using day 119 pooled antisera from cows immunized with the N. caninum killed vaccine. Similarly, in lanes 3 and 8 of FIG. 2, no reactivity against either form of the BAG1-GST protein wad detected using day 119 pooled antisera from cows immunized with the N. caninum attenuated vaccine.

The difference in reactivity to the T. gondii recombinant bradyzoite antigen described in this invention demonstrates that a serological test using a bradyzoite antigen can be used to discriminate or diagnose a N. caninum naturally infected versus N. caninum vaccinated animal. Any purified, native or recombinant bradyzoite antigen or fragment thereof, from N. caninum (e.g. SEQ ID No. 1 or 2) or N. caninum (e.g. SEQ ID No. 3or 4) can be used as the detection antigen for this diagnostic test. Any antisera from N. caninum naturally infected or N. caninum vaccinated animals can be used as the test sample for this diagnostic test.

1. A method for distinguishing an animal naturally seropositive for N. caninum from an animal vaccinated with a Neospora vaccine comprising obtaining a serum sample from the animal and contacting the serum with a Toxoplasma gondii protein and examining the serum for the presence of bound antibody.

2. The method of claim 1 wherein said Toxoplasma gondii protein comprises the amino acid sequence of SEQ ID NO:2.

3. The method of claim 1 wherein said Toxoplasma gondii protein comprises the amino acid sequence of SEQ ID NO:3.

4. The method of claim 2 wherein said Toxoplasma gondii protein is BAG-1.

5. The method of claim 2 wherein said Toxoplasma gondii protein is BAG-5

6. The method of claim 1 wherein the protein is attached to a support.

7. The method of claim 1 wherein the step of examining the protein for the presence of bound antibody is conducted by a Western Blot assay.

8. The method of claim 1 wherein the step of examining the protein for the presence of bound antibody is conducted by a radioimmunoassay.

9. The method of claim 1 wherein the step of examining the protein for the presence of bound antibody is conducted by an enzyme-linked immunosorbent assay.

10. The method of claim 1 further comprising quantifying the amount of antibody bound to the protein.

11. A method for diagnosing an animal suspected of having been infected with N. caninum comprising obtaining a serum sample from the animal and contacting the serum with a Toxoplasma gondii protein and examining the serum for the presence of bound antibody.

12. The method of claim 11 wherein said Toxoplasma gondii protein comprises the amino acid sequence of SEQ ID NO:2.

13. The method of claim 11 wherein said Toxoplasma gondii protein comprises the amino acid sequence of SEQ ID NO:3.

14. The method of claim 12 wherein said Toxoplasma gondii protein is BAG-1.

15. The method of claim 12 wherein said Toxoplasma gondii protein is BAG-5

16. The method of claim 11 wherein the protein is attached to a support.

17. The method of claim 11 wherein the step of examining the protein for the presence of bound antibody is conducted by a Western Blot assay.

18. The method of claim 11 wherein the step of examining the protein for the presence of bound antibody is conducted by a radioimmunoassay.

19. The method of claim 11 wherein the step of examining the protein for the presence of bound antibody is conducted by an enzyme-linked immunosorbent assay.

20. The method of claim 11 further comprising quantifying the amount of antibody bound to the protein.

21. A method for detecting antibodies to Neospora caninum in a serum sample comprising contacting the serum with a Toxoplasma gondii protein and examining the serum for the presence of bound antibody.

22. The method of claim 21 wherein said Toxoplasma gondii protein comprises the amino acid sequence of SEQ ID NO:2.

23. The method of claim 21 wherein said Toxoplasma gondii protein comprises the amino acid sequence of SEQ ID NO:3.

24. The method of claim 22 wherein said Toxoplasma gondii protein is BAG-1.

25. The method of claim 22 wherein said Toxoplasma gondii protein is BAG-5

26. The method of claim 21 wherein the protein is attached to a support.

27. The method of claim 211 wherein the step of examining the protein for the presence of bound antibody is conducted by a Western Blot assay.

28. The method of claim 21 wherein the step of examining the protein for the presence of bound antibody is conducted by a radioimmunoassay.

29. The method of claim 21 wherein the step of examining the protein for the presence of bound antibody is conducted by an enzyme-linked immunosorbent assay.

30. The method of claim 21 further comprising quantifying the amount of antibody bound to the protein.

31. A method for distinguishing an animal naturally seropositive for N. caninum from an animal vaccinated with a Neospora vaccine comprising obtaining a serum sample from the animal and contacting the serum with a Toxoplasma gondii protein fragment and examining the serum for the presence of bound antibody.


SEQUENCE LISTING (4 sequences)

SEQ ID NO: 1 - DNA, 1220 bp, Toxoplasma gondii
  CDS (219)..(905)
  CDS (303)..(905)
tgcttttgccaaaggagacctgtgtgcagaggacttcgctgctaaaaagcagaagagtgcacggcgtgtgtagctcagtggcatttcgggactcggtcttgcgtcgttcgcgactggacgtcgtcgtctgtgagagcgtcaaactagggagaaggggcgggccagagcgttcggaaaattatctgcaaagcccaggtcccgtatgatattcaaaaaagatggcgccgtcagcatcgcatccgccgggagcttgtccaccgggatgtaccaagcatcctgctactgcgacagccatttccccgagtggagtgtgtcccatgcgtgcgttccatcccgcggggcctcactcacatttctcatgttacgatgatctcagaaataggctgagtcacgacaaaaatgttcgaccggtcgcctctcaacagctagactatttggatgaagtaagcccgtttgctctggcgtactaccctcccccgttttggggtggagtcggtcttaatcccatcgacgatatgttgttcgagacggccctcaccgcaaacgaaatgatggaggacatcacgtggagacccagagtcgacgtggagttcgacagcaaaaagaaggaaatgattattttggctgacttgccaggtcttcagaaagatgacgtaaccatagaagtcgacaacggagccatcgttatcaaaggagagaagacctcgaaagaagcggagaaagtggacgatggcaaaacaaagaacattttgactgagcgagtgtccggttattttcgcgcccggttccagctcccgagtaattacaagcccgacggaatcagtgcggcaatggacaacggcgttctacgtgtcacgatcaaggtcgaggattcagggggcgcaaagcaacaaatcagcgtgaagtagaggcagcgatgccgttcgtggggcagggaaacacggaggagcctcattgaaaatgtaaaggtgtgggagaactgttgacagtgcaagataatagtacgagtaggattgcaaagaaggcctcccgtttctggcgccagccgagaaaacctggttgatgggcagcctcatgcgtgacggtttttctttagaggacttgtccgtcgaggtagtgcgtatcgcgtgtctgcggtttatcgtcacagcgcgtatccagaaacctagcgttcaacggctcaagaccgtaagcgaattagagttgaataggtgtgttat

SEQ ID NO: 2 - protein, 229 aa, Toxoplasma gondii
MAPSASHPPGACPPGCTKHPATATAISPSGVCPMRAFHPAGPHSHFSCYDDLRNRLSHDKNVRPVASQQLDYLDEVSPFALAYYPPPFWGGVGLNPIDDMLFETALTANEMMEDITWRPRVDVEFDSKKKEMIILADLPGLQKDDVTIEVDNGAIVIKGEKTSKEAEKVDDGKTKNILTERVSGYFRARFQLPSNYKPDGISAAMDNGVLRVTIKVEDSGGAKQQISVK

SEQ ID NO: 3 - protein, 201 aa, Toxoplasma gondii
SGVCPMRAFHPAGPHSHFSCYDDLRNRLSHDKNVRPVASQQLDYLDEVSPFALAYYPPPFWGGVGLNPIDDMLFETALTANEMMEDITWRPRVDVEFDSKKKEMIILADLPGLQKDDVTIEVDNGAIVIKGEKTSKEAEKVDDGKTKNILTERVSGYFRARFQLPSNYKPDGISAAMDNGVLRVTIKVEDSGGAKQQISVK

SEQ ID NO: 4 - DNA, 672 bp, Neospora caninum
  misc_feature (471)..(471): n=inosine
cccctttcttctctccgtttcaagatgagcaagctcagcacagacggcctgaagaaggccatcggagagatcttggagggctcgcgggaaaagaagagaaagttcgtggagaccgtcgagcttcagatcggtttgaaggattacgacacccaaagagacaagcgtttcagcggaagcgtgaggcttcctaacgtcccccgcccccgcatgcgcgtgtgcgtgatgggagatgctgtgcattgcgagcaagcaaaggagctgggactggaattcatggacgtggaggctatgaagaagttgaacaagaacaagaagcttgtgaagaaactcgcacggaagtacgacgctttcctggcctcgcaagtgttgattccgcagatcccccgtctcctgggtccgggtcttaacaaggcgggcaaattcccgacgcttatcactcacaacgataagctcgaggataagattcagganatcaagagctcaatcaaattccaactcagaaaggtgcctgtgcatggtgtcgccgtcnggcacgtcgacatgacggaggagcaactgcgcgtgaacctgacactggccatcaacttccttgtctctctgctgaagaagaactggaacagcgtgagaacgctgcacatcaagagcaccatgggcaagcctcagcagatttac